(19) United States
(12) Patent Application Publication
(10) Pub. No.: US 2002/0052318 A1
(43) Pub. Date: May 2, 2002

(54) Adjuvant

(75) Inventors: Barton F. Haynes, Durham, NC (US); Hua-Xin Liao, Chapel Hill, NC (US); Dhavalkumar D. Patel, Durham, NC (US)

Correspondence Address: NIXON & VANDERHYE P.C., 8th Floor, 1100 North Glebe Road, Arlington, VA, 22201, US

(73) Assignee: DUKE UNIVERSITY

(21) Appl. No.: 09/938,831

(22) Filed: Aug. 27, 2001

Related U.S. Application Data
(60) Provisional application No. 60/227,624, filed on Aug. 25, 2000.

(51) Int. Cl. A61K 48/00; A61K 38/17

(52) U.S. Cl. 514/12; 514/44; 514/8; 514/54

(57) ABSTRACT

The present invention relates, in general, to an adjuvant and, in particular, to an adjuvant that induces durable systemic and mucosal humoral, Th and/or CTL responses. The invention further relates to a composition, e.g., an immunogenic composition, comprising an immunogen and the adjuvant of the invention, and to a method of enhancing an immune response to an immunogen or immunogens using such an adjuvant.

This application claims priority from U.S. Prov. Appln. No. 60/227,624, filed Aug. 25, 2000, which is incorporated herein in its entirety by reference.

### TECHNICAL FIELD

The present invention relates, in general, to an adjuvant and, in particular, to an adjuvant that induces durable systemic and mucosal humoral, Th and/or CTL responses. The invention further relates to a composition, e.g., an immunogenic composition, comprising an immunogen and the adjuvant of the invention, and to a method of enhancing an immune response to an immunogen or immunogens using such an adjuvant.

### BACKGROUND

The adjuvant 3-O-deacylated monophosphoryl lipid A (MPL) both quantitatively and qualitatively enhances the antibody response to a wide range of bacterial and viral immunogens in experimental animals and man (Ulrich et al, The adjuvant activity of monophosphoryl lipid A. In Topics in Vaccine Adjuvant Research. D. R. Spriggs, and W. C. Koff, eds, CRC Press, Boca Raton, p. 13. (1991), Gordon et al, J. Infect. Dis. 171(6):1576-85 (1995), Heppner et al, J. Infect. Dis. 174(2):361-6 (1996), Van Hoecke et al, Vaccine 14(17-18):1620-6 (1996), Stoute et al, New Engl. J. Med. 336(2):86-91 (1997)). MPL is used as an aqueous solution, or as a stabilized oil-in-water emulsion (stable emulsion or SE) (the oil-in-water emulsion containing squalene, glycerol and phosphatidyl choline). The adjuvant activity of MPL has been attributed to the slow release of antigen to antigen-presenting cells (Ulrich et al, The adjuvant activity of monophosphoryl lipid A. In Topics in Vaccine Adjuvant Research. D. R. Spriggs, and W. C. Koff, eds, CRC Press, Boca Raton, p. 13. (1991)). Granulocyte-macrophage colony stimulating factor (GM-CSF) enhances both humoral and cell-mediated immunity when used as an adjuvant with protein immunogens (Ahlers et al, J. Immunol. 158(8):3947-58 (1997), Disis et al Blood 88(1):202-10 (1996), U.S. Pat. No. 5,078,996). It has been found that the antibody responses to peptides formulated in MPL can be enhanced by addition of GM-CSF (WO 00/69456).

Alpha-2-macroglobulin ((α₂M) is present in abundance in plasma and other body fluids, and is produced in many cell types, including macrophages. α₂M has the capacity, under certain conditions, to irreversibly capture diverse proteins for rapid delivery into macrophages (Chu et al, J. Immunol. 152:1538 (1994), Chu et al; J. Immunol. 150:48 (1993)) and it enhances the immunogenicity of proteins for antibody responses (Chu et al, J. Immunol. 152:1538 (1994), Chu et al; J. Immunol. 150:48 (1993)). α₂M consists of four identical subunits arranged to form a cage-like molecular “trap”. This trap is sprung when proteolytic cleavage within a highly susceptible stretch of amino acids, the “bait region”, initiates an electrophoretically detectable conformational change that entraps the proteinase (Swenson et al, J. Biol. Chem. 254:4452 (1979), Sottrup-Jensen et al, FEBS Lett. 127:167 (1981), Feldman et al, PNAS 82(17):5700-4 (1985), Salvesen et al,. Biochem. J. 195(2):453-61 (1981), Salvesen et al, Biochem. J. 187(3):695-701 (1980)). In addition to being non-covalently trapped, lysine-containing proteinases can spontaneously form covalent linkages by nucleophilic substitution at a thiolester located in each of the α₂M subunits. This thiolester becomes highly reactive during the conformational transition from native α₂M to the more compact, activated α₂M * form, (Salvesen et al,. Biochem. J. 195(2):453-61 (1981), Salvesen et al, Biochem. J. 187(3):695-701 (1980)). The resulting receptor-recognized α₂M * is efficiently internalized by macrophages and other cells that express α₂M * receptors. α₂M * receptor recognition is highly conserved for species as distantly related to mammals as the frog. The binding of nonproteolytic proteins to α₂M * does not affect the rate of internalization of the α₂M *-complex (Gron et al, Biochem. 37:6009 (1998)). Therefore, regardless of the mechanism of binding, any proteins coupled to α₂M * can be effectively internalized into antigen presenting cells (APC) such as macrophages and dendritic cells (DCs) (Chu et al, J. Immunol. 152:1538 (1994), Chu et al; J. Immunol. 150:48 (1993)).

Chu et al. found that injection of hen egg lysozyme (HEL) coupled to α₂M* generated 500-fold higher IgG titers in rabbits compared to the uncoupled control and the response was comparable to the response obtained with HEL in CFA (Chu et al, J. Immunol. 150:48 (1993)). It has been recently demonstrated that high levels of incorporation of various antigens into α₂M * prepared from human (Gron et al, Biochem. 37:6009 (1998)), mouse (Bhattacharjee et al, Biochem. Biophys. Acta 1432:49 (1999)) and rabbit can be achieved by a non-proteolytic activating method. (WO99/50305 describes a method for the preparation of a covalent complex between α₂M and an antigen that avoids the use of proteolytic enzymes.)

A major goal in the development of immunogenic compositions directed against human immunodeficiency type 1 (HIV-1) is to design a composition that will induce broadly reactive anti-HIV-1 humoral (antibody) and cellular (cytotoxic T lymphocyte; CTL) responses. For a polyvalent immunogen based on the variable regions of HIV envelope that induce neutralizing antibodies, many different HIV envelope peptides may need to be included to induce antibodies that neutralize sufficient primary isolates to be clinically relevant. For CTL induction, both dominant and subdominant CTL peptide epitopes can be combined in a sufficiently immunogenic formulation such that high levels of anti-HIV-1 CTL are induced (Haynes, Lancet 348:933 (1996), Ward et al, Analysis of HLA frequencies in population cohorts for design of HLA-based HIV vaccines. HIV Molecular Database. B. Korber, C. Brander, B. Walker, R. Koup, J. Moore, B. F. Haynes, G. Myers (Editors). Theoretical Biology Group, Los Alamos National Laboratory, Los Alamos, N. Mex., ppIV10-IV16 (1995)). To overcome the issue of HIV-1 variability, additional variant peptide epitopes at each CTL determinant can be included in the immunogen design. Such strategies can require the inclusion of approximately 50-100 different peptides in an immunogen.

The best adjuvant for use with peptides to induce specific antibody and CTL generation in animals is complete and incomplete Freund's adjuvant (CFA/IFA). However, the use of CFA/IFA is not permitted in humans. Furthermore, peak antibody and CTL responses are generally induced (in mice) with ˜100 μg of peptide immunogen formulated in CFA/IFA (Hart et al, PNAS 88:9448-52 (1991), Haynes et al, AIDS Research & Human Retroviruses. 11(2):211-21 (1995)). Thus, in order to develop a peptide immunogen based on as many as 50-100 different HIV-1 epitopes, it is desirable to reduce the dose of each individual peptide needed for immunization by at least 50-100 fold.

The present invention results, at least in part, from studies demonstrating that incorporation of HIV-1 envelope gp120 peptides into α₂M * can be used to augment HIV-1 peptide immunogenicity. Moreover, formulation of the α₂M *-HIV envelope complex in MPL-SE/GM-CSF results in an antigen formulation that is up to 100-fold more immunogenic than when the same HIV antigen is formulated in CFA/IFA.

### SUMMARY OF THE INVENTION

The present invention relates generally to adjuvants and more particularly to an adjuvant that induces durable systemic and mucosal humoral, Th and/or CTL responses and that is suitable for use, for example, in a multivalent immunogenic composition against HIV. The adjuvants of the invention are tolerated well regarding reactogenicity and provide a 1-2 log decrease in the antigen dose required to achieve a given level of immunogenicity.

Objects and advantages of the present invention will be clear from the description that follows.

### BRIEF DESCRIPTION OF THE DRAWINGS

FIG. 1. CTL response to C4-V3_{IIIB} peptide in Balb/c immunized with C4-V3_{IIIB} peptide. Mice were primed and boosted SQ (subcutaneously) with C4-V3_{III} peptides at the indicated amounts with MPL-SE (25 μg)/GM-CSF (10 μg) (solid box), α₂M * (empty box) or α₂M * plus MPL-SE (25 μg)/GM-CSF (10 μg) (hatched box) as adjuvant at day 0, 14, and 28. Ten days after the final immunization, immune spleen cells were harvested and restimulated in vitro with peptide in the presence of IL-2. Data shown are the results obtained at an E:T (effector:target) ratio of 10:1. All data are expressed as the mean of the values obtained from three mice.

FIG. 2. Serum antibody response to C4-V3_{IIIB} peptide in Balb/c immunized with C4-V3_{IIIB} peptide. Mice were primed and boosted SQ with C4-V3_{III} peptides at the indicated amounts with MPL-SE (25 μg)/GM-CSF (10 μg) (solid box), α₂M * (empty box) or α₂M * plus MPL-SE (25 μg)/GM-CSF (10 μg) (hatched box) as adjuvant at day 0, 14, and 28. Serum samples were collected seven days after the final immunization, and assayed against C4-V3_{III} peptide in a ELISA assay. The antibody end-point binding titers were determined as the reciprocal of the highest dilution of the serum assayed against immunizing peptide giving OD (optical density) reading of experiment/control of >3.0. The log end-point titers of serum samples are presented in the Y-axis.

FIG. 3. CTL response to C4-V3_{IIIB} peptide in Balb/c immunized with C4-V3_{IIIB} peptide. Mice were primed and boosted with 10 μg C4-V3_{III} peptides using alum, α₂M *, MPL-SE (25 μg)/GM-CSF (10 μg), or α₂M * plus MPL-SE/GM-CSF as adjuvant, as indicated, at day 0, 14, and 28. The immune spleen cells were harvested 10 days after the final immunization, and restimulated in vitro with peptide in the presence of IL-2. Data shown are the results obtained at an E:T ratio of 80:1, 40:1, 20:1, and 10:1. All data are expressed as the mean of the values obtained from three mice.

FIG. 4. Schematic representation of steps in generating and amplifying an immune response induced by an immunogenic composition.

FIG. 5. Serum antibody response to C4-V3_{IIIB} peptide in Balb/c immunized with C4-V3_{IIIB} peptide. Mice were immunized with 25 μg C4-V3_{III} peptide using IFA, MPL-SE (25 μg)/GM-CSF (10 μg), MPL-SE/GM-CSF+TARC (0.1 μg), MPL-SE/GM-CSF+ELC (0.1 μg), MPL-SE/GM-CSF+LARC (0.1 μg), MPL-SE/GM-CSF+MDC (0.1 μg), or MPL-SE/GM-CSF+TARC, ELC, LARC and MDC as adjuvant at day 0, 14, and 28. Serum samples were collected seven days after the final immunization, and assayed against C4-V3_{III} peptide in a ELISA assay. The antibody end-point binding titers were determined as the reciprocal of the highest dilution of the serum assayed against immunizing peptide giving OD reading of experiment/control of >3.0. The log end-point titers of serum samples are presented in the Y-axis.

FIG. 6. Serum antibody response to C4-V3_{IIIB} peptide in Balb/c immunized with C4-V3_{IIIB} peptide. Mice were immunized with 25 μg C4-V3_{III} peptide using IFA, α₂M *, α₂M *+Alum+TARC (0.1 μg), α₂M *+Alum+ELC (0.1 μg), α₂M *+Alum+LARC (0.1 μg), α₂M *+Alum+MDC (0.1 μg), α₂M *+Alum+TARC, ELC, LARC and MDC, or α₂M *+Alum+TARC, ELC, LARC, MDC and MPL-SE/GM-CSF as adjuvant as indicated at day 0, 14, and 28. Serum samples were collected seven days after the final immunization, and assayed against C4-V3III peptide in a ELISA assay. The antibody end-point binding titers were determined as the reciprocal of the highest dilution of the serum assayed against immunizing peptide giving OD reading of experiment/control of >3.0. The log end-point titers of serum samples are presented in the Y-axis.

FIG. 7. Serum antibody response to C4-V3_{IIIB} peptide in Balb/c immunized with C4-V3_{IIIB} peptide. Mice were immunized with C4-V3_{III} peptide at the indicated amounts using IFA, MPL-SE/GM-CSF, α₂M *, α₂M *+Alum, or, α₂M *+Alum+MPL-SE/GM-CSF+TARC, ELC, LARC and MDC as adjuvant as indicated at day 0, 14, and 28. Serum samples were collected seven days after the final immunization, and assayed against C4-V3_{III} peptide in a ELISA assay. The antibody end-point binding titers were determined as the reciprocal of the highest dilution of the serum assayed against immunizing peptide giving an OD reading of experiment/control of >3.0. The log end-point titers of serum samples are presented in the Y-axis.

FIGS. 8A and 8B. Isotype of the C4-V3_{IIIB}-reactive antibody in animals immunized with α₂M *-coupled C4-V3_{IIIB} peptide. Antisera from mice receiving 10 μg of C4-V3 peptide coupled to α₂M*. (FIG. 8A), or 100 μg of C4-V3_{IIIB} peptide using CFA/IFA (FIG. 8B) as adjuvant were assayed against C4-V3_{IIIB} peptide by ELISA to determined the major immunoglobulin isotypes of the antibody reactive to C4-V3_{IIIB} peptide. The antibody end-point binding titers were determined as the reciprocal of the highest dilution of the serum assayed against immunizing peptide giving OD reading of experiment/control of ≧3.0, and shown on the y axis. The x axis shows the anti-isotype specific secondary antibodies used in the ELISA assay.

FIG. 9. Specificity of antibody response induced by α₂M*-coupled C4-V3_{IIIB} peptide. Serum samples were collected from mice before and after immunization with 10 μg α₂M*-coupled C⁴-V3_{IIIB}, and assayed against the saturated amounts of α₂M*, α₂M*-coupled HBsAg, or α₂M*-coupled C4-V3_{IIIB} peptide captured on 96-well ELISA plates as indicated on the right-handed side of the Figure. The x axis shows the a two-fold serial dilution of serum samples (n=3). The vertical axis shows the ELISA absorbance at wavelength of 405 nm OD on the right-handed side of the Figure. Data represent mean value±SEM OD at 405 nm of serum samples

FIG. 10. Serum antibody response to C4-V3_{IIIB} peptide in Balb/c immunized with C4-V3_{IIIB} peptide. Mice were primed and boosted SQ with C4-V3_{IIIB} peptides at the indicated amounts with MPL-SE (25 μg)/GM-CSF (10 μg) (solid columns), α₂M*-HIV peptide (hatched box) or α₂M*-HIV peptide plus MPL-SE (25 μg)/GM-CSF (10 μg) (empty columns) as adjuvant at day 0, 14, and 28. Serum samples were collected seven days after the final immunization, and assayed against C4-V3_{IIIB} peptide in an ELISA assay. The antibody end-point binding titers were determined as the reciprocal of the highest dilution of the serum assayed against immunizing peptide giving OD reading of experiment/control of ≧3.0. The log end-point titers of serum samples are presented in the Y-axis.

FIG. 11. Duration of serum antibody response against C4-V3_{IIIB} peptide. Mice were primed and boosted SQ with C4-V3_{IIIB} peptides at the indicated amounts of peptide using adjuvant formulation of α₂M*+MPL-SE/GM-CSF or MPL-SE/GM-CSF alone on day 0, 14, 28 as indicated by arrows. Serum samples were collected on day 0, 35, and 184, and assayed against C4-V3_{IIIB} peptide in an ELISA assay. The antibody end-point binding titers were determined as the reciprocal of the highest dilution of the serum assayed against immunizing peptide giving OD reading of experiment/control of ≧3.0. The log ELISA end-point titers of serum sample are presented in the Y-axis.

FIG. 12. Serum antibody response in Balb/c immunized with C4_{E9V}-V3.89._{6P} peptide. Mice were primed and boosted SQ with 100 μg. 10 μg, 5 μg, 1 μg, 0.5 μg, or 0.1 μg of C4_{E9V}-V3 _{89.6P} peptides as indicated using α₂M*-HIV peptide, MPL-SE/GM-CSF, or α₂M*-HIV peptide plus MPL-SE/GM-CSF as adjuvant at day 0, 14, and 28. Serum samples were collected seven days after the final immunization, and assayed against C4_{E9V}-V3 _{89.6P} peptide in an ELISA assay. The antibody end-point binding titers were determined as the reciprocal of the highest dilution of the serum assayed against immunizing peptide giving OD reading of experiment/control of ≧3.0. The log end-point titers of serum samples are presented in the Y-axis.

FIGS. 13A and 13B. CTL response to C4-V3_{IIIB} peptide in Balb/c immunized with C4-V3_{IIIB} peptide. FIG. 13A. Mice were primed and boosted with 100 μg, 50 μg, 10 μg, 1 μg and 0.5 μg C4-V3_{IIIB} peptide using MPL-SE/GM-CSF (FIG. 13A, solid columns) as adjuvant, or with lower dose of 10 μg, 5 μg, 1 μg, 0.5 μg and 0.1 μg C4-V3_{IIIB} peptides using α₂M*-HIV peptide alone (FIG. 13A, hatched columns), or α₂M*-HIV peptide plus MPL-SE/GM-CSF (FIG. 13A, empty columns) as indicated at day 0, 14, and 28. The immune spleen cells were harvested 10 days after the final immunization, and restimulated in vitro with peptide in the presence of IL-2. Data shown are the results obtained at an E:T ratio of 10:1. FIG. 13B. Comparison of CTL responses in mice primed and boosted with 10 μg C4-V3_{IIIB} peptide using MPL-SE/GM-CSF alone, α₂M*-HIV peptide alone, or α₂M*-HIV peptide plus MPL-SE/GM-CSF as adjuvant. Data shown are the results obtained at an E:T ratio of 80:1, 40:1, 20:1, and 10:1. All data are expressed as the mean of the values obtained from three mice.

### DETAILED DESCRIPTION OF THE INVENTION

The present invention relates to a novel adjuvant suitable for use, for example, in multivalent immunogenic compositions, including multivalent HIV immunogenic compositions. The invention is based, at least in part, on the finding that combining activated alpha-2-macroglobulin (α₂M*) “loaded” with HIV peptides with MPL/GM-CSF provides better immunogenicity than either α₂M* or MPL/GM-CSF alone, thereby lowering the required dose of HIV peptide. The invention is further based on the finding that the addition one or more of chemokines, for example, TARC (Thymus and Activation Regulated Chemokine), ELC (Epstein-Barr Virus-induced molecule 1 (EBI-1) Ligand Chemokine), LARC (Liver and Activation Regulated Chemokine), BLC (B Lymphocyte Chemokine) and/or MDC (Macrophage Derived Chemokine) to MPL/GM-CSF/α₂M* can additionally lower the required dose of HIV peptide.

Generally, the compositions of the invention comprise an immunogen(s) and an adjuvant, wherein the adjuvant comprises α₂M*; α₂M* plus MPL/GM-CSF; α₂M* plus at least one chemokine or B cell activator/growth factor; MPL/GM-CSF plus at least one chemokine or B cell activator/growth factor (with or without α₂M*); α₂M* plus at least one angiogenic factor; or MPL/GM-CSF plus at least one angiogenic factor (with or without α₂M*). MPL can be present as an aqueous solution or as a stabilized oil-in-water emulsion (stable emulsion or SE) (the oil-in-water emulsion comprising, for example, squalene, glycerol and phosphatidyl choline) (see WO 00/69456). Any of the compositions can further comprise a cytokine (e.g., a cytokine other than GM-CSF). The compositions can also include an immunologically acceptable diluent or carrier. The optimum concentration of each component of any of the compositions can be readily determined by one skilled in the art (see WO 00/69456 and WO 99/50303). The compositions can be used to generate a therapeutic or prophylactic effect in a human or non-human vertebrate.

In one embodiment, the present invention relates to a method of enhancing B cell antibody responses and T cell helper and cytotoxic T cell responses to an immunogen (e.g., a peptide, polypeptide or protein immunogen) using combinations of MPL/GM-CSF (or functionally comparable components, synthetic or naturally occurring) and α₂M* loaded with the immunogen, with or without one or more chemokines, for example, TARC, LARC, ELC, BLC or MDC, and with or without one or more cytokines, for example, IL-2, IL-15, IL-7 or IL-12. The α₂M* can be produced from native plasma α₂M or can be recombinantly produced (see WO 99/50303 for details of immunogen loading of α₂M). The invention also relates to such combinations.

In a further embodiment, the present invention relates to a method of enhancing B cell antibody responses and T cell helper and cytotoxic T cell responses to an immunogen using combinations of MPL/GM-CSF (or functionally comparable components) with a chemokine, e.g., TARC, LARC, ELC, BLC and/or MDC, and with or without a cytokine, e.g., IL-2, IL-15, IL-7 and/or IL-12. The invention further relates to such combinations.

In another embodiment, the present invention relates to a method of enhancing B cell antibody responses to an immunogen using combinations of MPL/GM-CSF (or functionally comparable components) and one or more B cell activator/growth factors such as BLyS (a B lymphocyte stimulator—Moore et al, Science 285:260 (1999)) or APRIL (a proliferation-inducing ligand—Hahne et al, J. Exp. Med. 188:1185 (1998)), with or without the imunogen being complexed to α₂M*. The invention further relates to such combinations.

In yet another embodiment, the present invention relates to a method of enhancing B cell antibody responses, T cell helper responses and T cell cytotoxic T cell responses to an immunogen using mixtures of MPL/GM-CSF (or functionally comparable components) and an angiogenic factor, e.g., VEGF, bFGF and/or low MW (molecular weight) hyaluronan fragments. In such a mixture, the immunogen can be mixed with the above components or complexed with α₂M* in the mixture (i.e., with or without α₂M*). The invention further relates to such mixtures.

Sandberg et al have demonstrated that one cause of the immune system selecting dominant over non-dominant epitopes for CTL recognition is T cell competition for DCs, too few DCs to present all available T cell epitopes (J. Immunol. 160:3163-3169 (1998)). In their system, non-dominance was overcome by purifying DCs, antigen pulsing DCs in vitro, and immunizing with pulsed DCs. When this was done, non-dominant CTL epitopes became dominant (J. Immunol. 160:3163-3169 (1998)). It has recently been shown that not all DCs can present antigen (rev. in Lanzavecchia, Nature 393:413-414 (1998)). Those that are “resting” or “immature” (iDCs) and do not express high levels of CD40 and other costimulatory molecules are inefficient presenters of antigen (Lanzavecchia, Nature 393:413-414 (1998)). T helper cell activation of DCs occurs via inflammatory cytokines (GM-CSF, IFN-γ and TNFα) as well as via ligation of DCs with T cell surface CD40 ligand (CD40L) (Lanzavecchia, Nature 393:413-414 (1998)). Ligation of DC CD40 by CD40L or CD40 mabs and treatment of DCs by inflammatory cytokines leads to DC activation, upregulation of DC MHC molecules and induction of efficient antigen presentation activity. Triggering of DCs with TNFα leads to DC production of IL-12 that is a potent activator of CTL (Lanzavecchia, Nature 393:413-414 (1998)). The present compositions, as discussed below, serve to: i) attract immature DCs to the immunization site, and ii) induce maturation of and activate DCs to more potently activate CTL responses.

The importance of angiogenesis in inflammation is now being emphasized with the recent breakthroughs in understanding of the cellular and molecular nature of angiogenic and anti-angiogenic factors (Yancopoulos et al, Cell 93:661-664 (1998), Jackson et al, FASEB J. 11:457-465 (1997)). One angiogenic factor that is in human therapeutic trials is vascular endothelial growth factor (VEGF). It is currently being used to treat arterial insufficiency in a number of clinical settings (Aruffo et al, Cell 61:1303-1313 (1990)). The potency of any adjuvant is directly related to the degree of chronic inflammation that is induced, and there is a need to strike a balance in adjuvant development between immunogenicity and an “appropriate” degree of inflammation to lead to a durable and efficacious level of memory T cell induction. Too much inflammation can lead to systemic symptoms and local reactions that reach a clinically unacceptable level. In spontaneous chronic inflammation such as occurs in rheumatoid arthritis, or in adjuvant-induced inflammation, angiogenesis is likely very important in the initiation of the immune response with formation of high endothelial venules (HEV) that facilitate migration and extravasation of T, B, DCs and macrophages to the site of antigen deposition, such as the site of immunization.

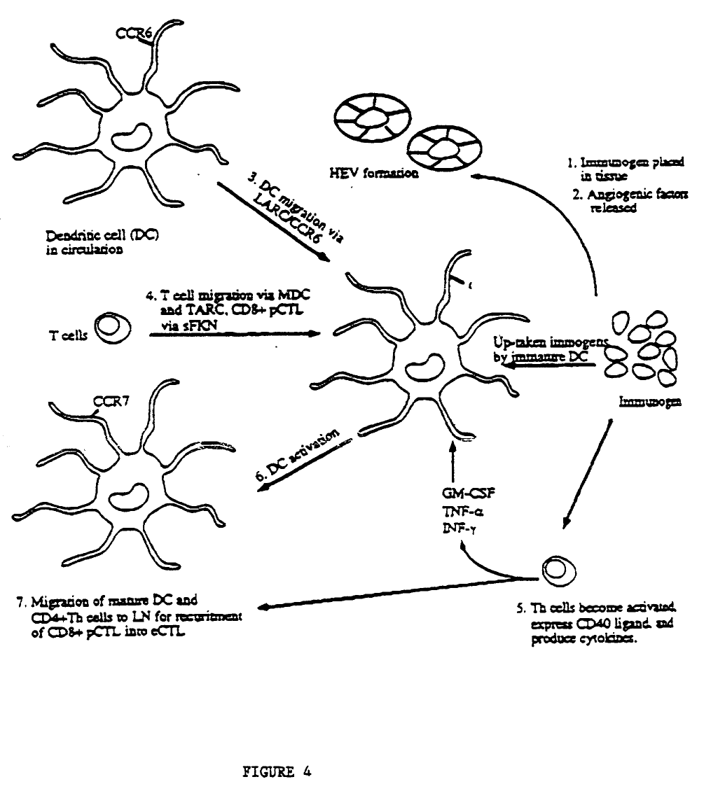

A schema can now be developed regarding postulated steps that occur at a cellular and molecular level in the immunization microenvironment at the site of a successful immunization (FIG. 4). Immunogen is deposited at the immunization site (Step 1), and angiogenic factors are induced from immunization site fibroblasts (Step 2). With the increase in blood vessels and formation of high endothelial venules, as well as production of chemoattractants such as LARC, dendritic cells and T cells migrate to at the site of immunization (Step 3). T cells migrate via chemokines, MDC (monocyte-derived chemokine) and TARC and CD8 pCTL migrate via fractalkine (FKN) to sites of inflammatory (Step 4) and to regional LN (Step 5). DCs phagocytose and process immunogen, down regulate CCR7 (Step 6) and home to regional LN to recruit more Th cells and present antigen to naive CD8+T cells (Step 7) (Hieshima et al, J. Biol. Chem. 272:5846 (1997), Imai et al, J. Biol. Chem. 271:21514 (1996), Godiska et al, J. Exp. med. 185:1595 (1997), Fong et al, B. Exp. Med. 188:1413-1419 (1998), Graves et al, J. Exp. Med. 186:837-844 (1997)). Th cells at the site of immunization and in the regional lymph nodes produce TNFα, GM-CSF and IFN-γ, all cytokines that induce DCs to mature (Step 5) (see FIG. 4). The steps that are outlined in FIG. 4 represent points at which this sequence of events can be acted upon to augment immunogenicity of an immunogen both systemically and mucosally and increase the memory CD8+T cell pool to HIV immunogens.

As shown in the Examples that follow, the combination of MPL-SE/GM-CSF/α₂M* and chemokines lowered the optimal dose of peptide from 100 μg for IFA or MPL-SE/GM-CSF to 1 μg with the combination. Recently, a new chemokine BLyS, also known as zTNF4 (z Tumor Necrosis Factor 4—Gross, Nature 404:995 (2000)), BAFF (B cell Activating Factor belonging to the TNF family—Schneider et al, J. Exp. Med. 189:1747 (1999)), TALL-1 (TNF- and Apol-related Leukocyte expressed Ligand 1—Shu et al, J. Leuk. Biol. 65:680 (1999))and THANK (TNF Homologue that Activates apoptosis, Nk-kB and JNK—Mukhopadhyay et al, J. Biol. Chem. 274:15978 (1999)), has been reported to stimulate B lymphocytes to make IgA (Hilbert, Human Genome Sciences, Inc., AAI meeting, Seattle, Wash., May, 2000)). Thus, addition of BLyS as well as other B cell activators/growth factors such as APRIL can be used to raise antigen-specific immunoglobulin levels, and in particular raise IgA levels. For infections that attack at mucosal surfaces, induction of high levels of pathogen IgA both systemically and at mucosal surfaces are important for induction of protective immunity.

The adjuvant formulations of the invention, including those set forth in Table 2 (wherein MPL can be present as an aqueous solution or as a stabilized oil-in-water emulsion (designated MPL-SE)), can be used to enhance immune responses (e.g., human immune responses) to any immunogen used to induce prophylactic or therapeutic immunity for any infectious disease pathogen, any cancer, or to manipulate immune responses with immunogens in any autoimmune disease. A preferred adjuvant formulation includes the combination of MPL/GM-CSF (e.g., MPL-SE/GM-CSF) (or functionally comparable components) plus α₂M* (e.g., human α₂M*) wherein the α₂M* is loaded with the immunogen of choice. While in studies described in the Examples the HIV envelop peptide C4-V3 immunogen is used, proteins up to ˜100 kilodaltons or more can be used to be bound to α₂M* for delivery.

TABLE 2
Combination of Adjuvant Components

MPL/GM-CSF + α₂M*
MPL/GM-CSF + α₂M* + TARC
MPL/GM-CSF + α₂M* + ELC
MPL/GM-CSF + α₂M* + LARC
MPL/GM-CSF + α₂M* + MDC
MPL/GM-CSF + α₂M* + BLC
MPL/GM-CSF + α₂M* + TARC + ELC + LARC + MDC
MPL/GM-CSF + TARC
MPL/GM-CSF + ELC
MPL/GM-CSF + LARC
MPL/GM-CSF + MDC
MPL/GM-CSF + BLC
MPL/GM-CSF + TARC + ELC + LARC + MDC
MPL/GM-CSF + VEGF + α₂M*
MPL/GM-CSF + bFGF + α₂M*
MPL/GM-CSF + low MW hyaluronan fragments + α₂M*
MPL/GM-CSF + VEGF + bFGF + + low MW hyaluronan fragments +
α₂M*
MPL/GM-CSF + VEGF
MPL/GM-CSF + bFGF
MPL/GM-CSF + low MW hyaluronan fragments
MPL/GM-CSF + VEGF + bFGF + low MW hyaluronan fragments
MPL/GM-CSF + α₂M* + BLyS
MPL/GM-CSF + α₂M* + APRIL
MPL/GM-CSF + α₂M* + BLyS + APRIL
MPL/GM-CSF + IL-2
MPL/GM-CSF + IL-7
MPL/GM-CSF + IL-12
MPL/GM-CSF + IL-15
MPL/GM-CSF + IL-2 + IL-12 + IL-15 + IL-7
MPL/GM-CSF + IL-2 + α₂M*
MPL/GM-CSF + IL-15 + α₂M*
MPL/GM-CSF + IL-7 + α₂M*
MPL/GM-CSF + IL-12 + α₂M*
MPL/GM-CSF + IL-2 + IL-15 + IL-7 + IL-12 + α₂M*
Any Mixture of the Above:
Eg MPL/GM-CSF, α₂M*, TARC, LARC, MDC, ELC, BLC, BLyS, IL-2
Note:
When α₂M* is mentioned it means that the immunogen (peptide, polypeptide or protein) has been covalently linked to α₂M*. When α₂M* is not in the mixture, the immunogen can be mixed with the MPL/GM-CSF in squalene. In addition, each of the cytokines/chemokines can be used alone or in combinations listed above with peptide/polypeptide/protein or polysaccharide immunogenic compositions or with DNA immunogenic compositions.

As indicated above, a series of chemokines, cytokines and/or angiogenic factors can be added to MPL/GM-CSF+α₂M*-antigen. The data provided in the Examples demonstrate that this adjuvant formulation is of use for HIV immunogens when the chemokines TARC, ELC, LARC and MDC are used. For induction of systemic and mucosal IgA levels of anti-pathogen antibodies and/or T helper cells and/or cytotoxic T cells, addition of BLys and/or APRIL can be important (Hilbert, Human Genome Sciences, Inc., AAI meeting, Seattle, Wash., May, 2000)). One skilled in the art will appreciate that optimal responses for, for example, different pathogen or cancer or autoimmune immunogenic compositions can vary, and different combinations of MPL/GM-CSF/α₂M*/and chemokines can be optimal for various immunogenic compositions. In addition, for enhancement of T cell responses, inclusion of IL-2, IL-7, IL-12 and/or IL-15 proteins with MPL/GM-CSF/α₂M*/chemokines can be used for maximum immunogenicity.

The cytokines (for example, IL-2, IL-7, IL-12, IL-15, BlyS, APRIL) or chemokines (for example, TARC, ELC, LARC, BLC) can be administered either as recombinant proteins or as plasmid DNAs as part of a DNA immunogenic composition (Seder et al, New Eng. J. Med. 341:277-278 (1999)). The cytokines and chemokines can be constructed as cytokine or chemokine/Ig fusion proteins or as part of a DNA construct so that the expressed cytokines or chemokine fusion protein is expressed as a protein with a long half-life. Since, as indicated above, increases in blood supply by induction of angiogenic factors is an early step in the adjuvant and inflammatory response (Yancopoulos et al, Cell 93:661-664 (1998), Jackson et al, FASEB J. 11:457-465 (1997), Aruffo et al, Cell 61:1303-1313 (1990)), the immunogenicity of MPL-SE/GM-CSF plus α₂M*-loaded with the desired antigen can be enhanced by the administration of angiogenic factors such as vascular endothelial growth factor (VEGF), basic fibroblast growth factor (bFGF), or low molecular weight hyaluronan fragments.

The adjuvants of the invention are suitable for use in immunogenic compositions containing a wide variety of immunogens from a variety of pathogens (including viruses, bacteria and fungi) or from cancer cells. Immunogens comprising insulin peptides can also be used in the compositions of the invention and administered for the prevention/treatment of diabetes. Examples of infectious agent immunogens that can be administered complexed with α₂M* or mixed with MPL/GM-CSF with chemokines and/or cytokines (with or without α₂M*) are: human immunodeficiency virus, influenza, mycobacterium, tuberculosis, Ebola virus, Hepatitis C, Hepatitis B, measles, mumps, polio, tetanus, and malaria proteins (see also immunogens described in WO/0069456).

In accordance with the methods of the present invention, the formulations/compositions can be administered by any of a variety of routes including, but not limited to, intramuscular, subcutaneous, intradermal, intranasal, vaginal, intravenous or oral. In the absence squalene-based MPL, α₂M*-immunogen, (e.g., plus cytokines or chemkines) can be administrated intranasally for optimum induction of mucosal immunity (Porgador et al, J. Immunol. 158:834-841 (1997)). The amount of the immunogen or immunogens in the composition will vary with the composition used, the patient, the route of administration and/or the effect sought. It is preferable, although not required, that all components of the immunogenic composition (immunogen and adjuvant (as well as chemokines and cytokines)) be administered at the same time. Optimum dosages and dosing regimens can be readily determined by one skilled in the art.

The adjuvant component(s) of the compositions of the invention can be formulated as recombinant proteins (e.g., as fusion proteins comprising chemokines or cytokines and/or immunogen) to be administered with MPL (or functionally comparable component)/GM-CSF and/or α₂M* loaded with the desired peptide, polypeptide or protein, or DNA encoding same, or any other immunogenic composition not complexed with α₂M*.

The adjuvants of the present invention are suitable for inclusion in nucleic acid-based (e.g., DNA-based) immunogenic compositions (see, for example, U.S. Pat. No. 5,593,972 and U.S. Pat. No. 5,589,466)

Certain aspects of the present invention are described in greater detail in the non-limiting Example that follows.

### EXAMPLE I

C4-V3_{IIIB} peptide was complexed with mouse α₂M*, and Balb/c mice were immunized for induction of antibody and antigen-specific CTL responses. Mouse α₂M* was highly effective and a potent adjuvant for induction of anti-HIV antibody response when the C4-V3_{IIIB} subunit immunogen was coupled to mouse α₂M* and administered subcutaneously (Table 1). Compared to the immunization with no adjuvant, specific antibody responses were induced with as low as 0.5 μg of C4-V3_{IIIB}. This amount of peptide is 100-fold lower than that required for induction of a similar antibody response by the same antigen with adjuvant. In comparison with the standard adjuvant CFA/IFA, α₂M* decreased the dose of peptide required for induction of maximal antibody response by approximately 10-fold, with maximal response achieved with 5 μg of peptide using α₂M* adjuvant. No antibodies were induced when lower than 50 μg of C4-V3_{IIIB} was used in CFA/IFA or the no adjuvant group (Table 1).

TABLE 1
Comparison of the ability of C4-V3 peptide to induce antibodies
in Balb/c mice using α2M* and CFA/IFA

| | Immunizing Peptide C4-V3IIIB | | | | | |
|---|---|---|---|---|---|---|
| Adjuvant | 50 μg | 10 μg | 5 μg | 1 μg | 0.5 μg | 0.1 μg |
| None | 330 | <50 | <50 | <50 | <50 | <50 |
| CFA + IFA | 9307 | <50 | <50 | <50 | <50 | <50 |
| α2M* | 4267 | 34433 | 6467 | 883 | 533 | <50 |

Data represent mean ELISA endpoint titers of three mouse sera as the reciprocal of the highest dilutions of serum samples at which the E/C was ≧ 3.0 in anti-immunizing peptide ELISA after three immunizations. Note: peptide was covalently coupled to α₂M* when α₂M* was used.

Using CFA/IFA, the dose of peptide for use in mice of an HIV experimental immunogenic composition is approximately 100 μg/dose in mice.

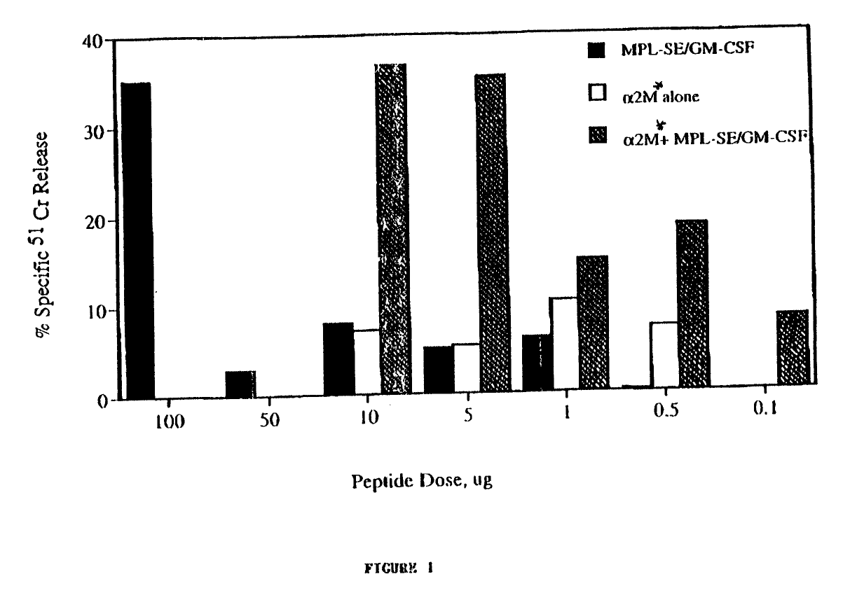

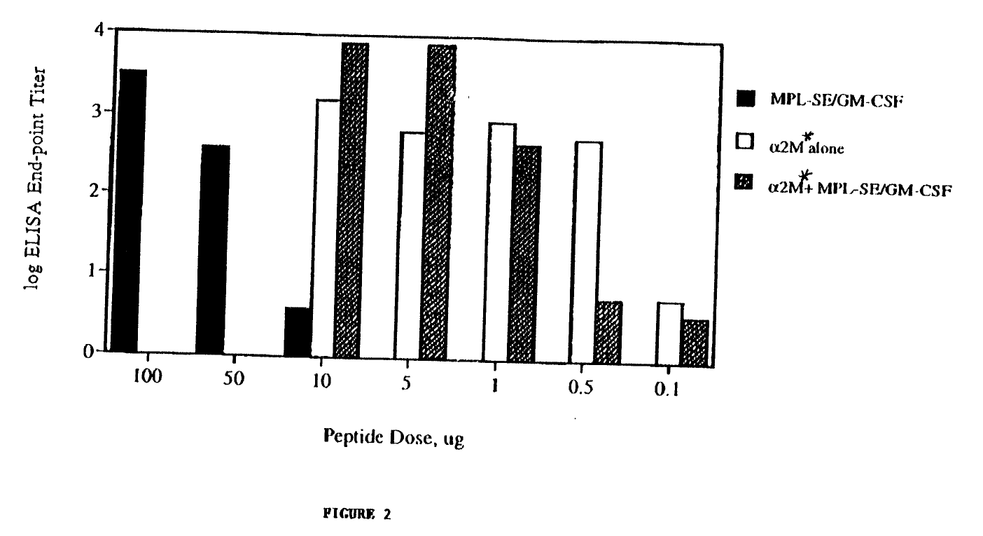

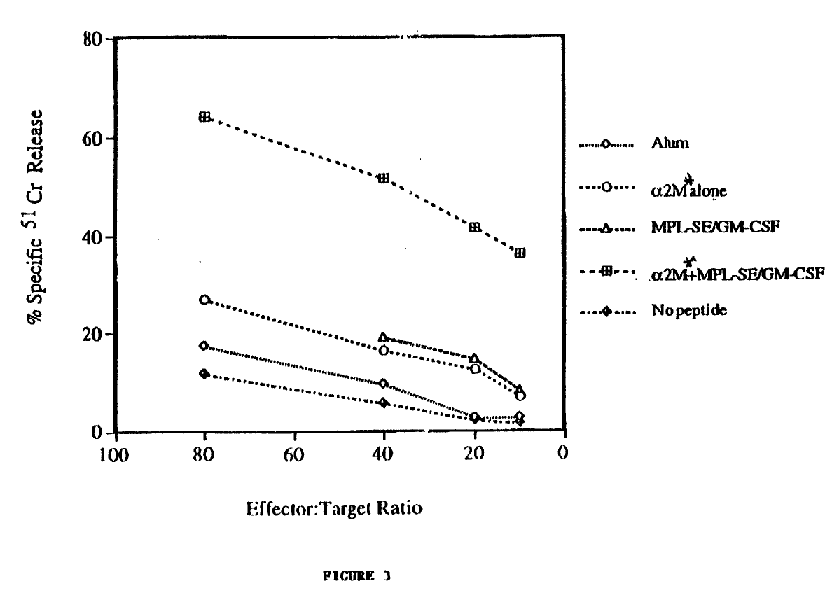

MPL-SE+GM-CSF (WO 00/69456) was used alone and mixed with other compounds to achieve a new series of adjuvants for use with HIV and other immunogens. Combining MPL-SE/GM-CSF with α₂M* “loaded” with HIV C4-V3 peptides gave better immunogenicity than either alone, with the peak dose being 5 μg with MPL-SE/GM-CSF+α₂M* together compared to 100 μg with MPL-SE/GM-CSF alone or 10 μg of α₂M* alone (FIG. 1). These same improvements in HIV peptide antigenicity were also seen for CTL generation (FIGS. 2, 3).

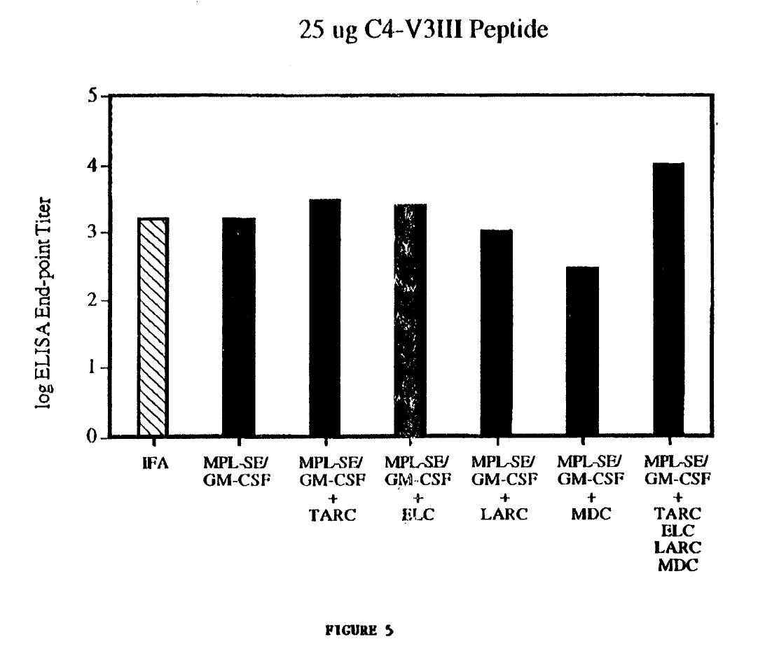

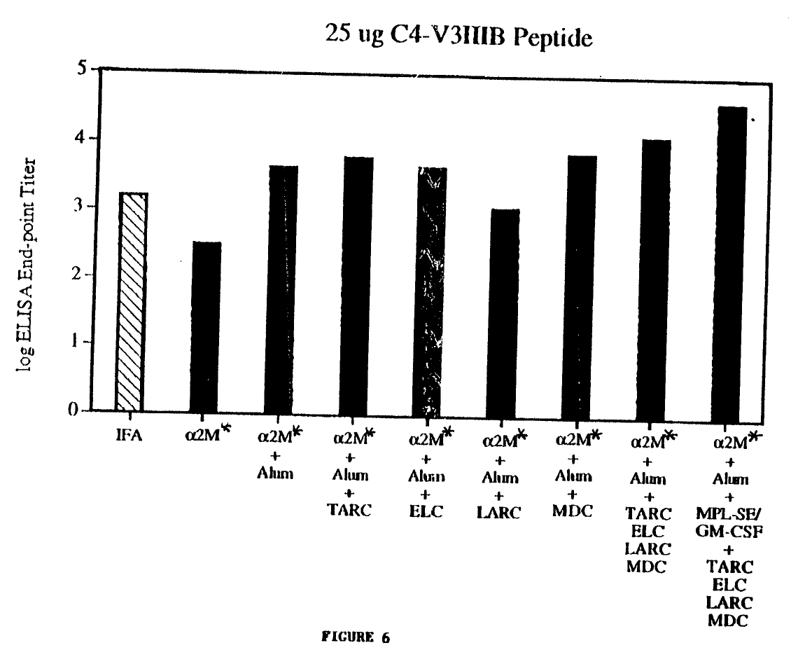

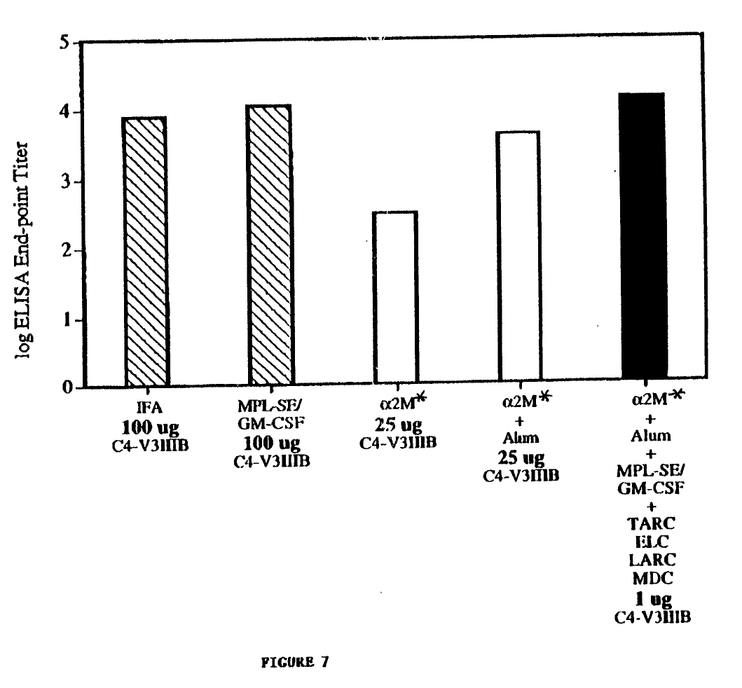

To take advantage of recent discoveries in the molecules that direct cell movement around the body, these key chemokines, thymus and activation regulated chemokine (TARC), EBL-1 ligand chemokine (ELC), liver and activation regulated chemokine (LARC) and MDC, were added to MPL-SE/GM-CSF/α₂M*. It was shown that the latter combination of chemokines, α₂M* and MPL-SE/GM-CSF lowered the optimal dose of peptide from 100 μg for IFA or MPL-SE/GM-CSF to 1 μg with the combination of chemokines/MPL-SE/GM-CSF/α₂M* (FIGS. 5-7).

### EXAMPLE II

### Experimental Details

Purification of mouse α₂-macroglobulin(α₂M): Aseptically collected, anti-coagulated (acid citrate dextrose; ACD) mouse plasma was obtained from Pel-Freez Biologicals (Rogers, Ariz.). All purification steps were performed at 4° C. using endotoxin-free plasma, columns and buffers as previously described (Chu et al, Journal of Immunology 152:1538-1545 (1994)). The purified proteins contained less than 100 pg/ml endotoxin as determined by a commercial assay kit (Kinetic QCL; BioWhittaker; Walkersville, Md.). Murine α₂M was prepared using the method as previously described (Chu et al, Journal of Immunology 152:1538-1545 (1994)). Briefly, diluted citrated mouse plasma was loaded on to a Cibracon Blue F-3GA agarose (Amersham Pharmacia Biotech; Piscataway, N.J.) affinity column pre-equilibrated with a buffer containing 100 mM NaCl and 20 mM HEPES, pH 7.4. The flow-through fractions contained the protein of interest as assayed by bovine trypsin inhibitory activity utilizing blue hide powder azure as a substrate. The α₂M-containing fractions were pooled, diluted and subjected to anion-exchange chromatography on a DEAE-Sephacel (Amersham Pharmacia Biotech). The column was developed with a linear gradient of NaCl from 0 to 400 mM in 20 mM HEPES, pH 7.4. Fractions containing the α₂M were pooled, concentrated, and subjected to gel-filtration chromatography on a Sephacryl S-300 HR (Amersham Pharmacia Biotech) column eluted with the above buffer. Those fractions containing α₂M were pooled and concentrated using CentriPrep® concentrators (Millipore; Bedford, Mass.).

HIV-1 envelope gp120 C4-V3 peptides: Synthetic peptides were synthesized by SynPep Corporation, Dublin, Calif., and purified by reverse phase HPLC. Peptides were greater than 95% pure as determined by HPLC, and confirmed by mass spectrometry. The V3_{IIIB} peptide (TRPNNNTRKSIRIQRGPGRAFVTI) was derived from the HIV-1 IIIB isolate (Chu et al, J. Immunol. 150:48-58 (1993)) and V3_{89.6P} (TRPNNNTRERLSIGPGRAFYARR) peptide from the pathogenic strain of SHIV-89.6P, clone KB-9 (Sandberg et al, J. Immunol. 160:3163-3169 (1998)). To enhance peptide immunogenicity, the V3_{IIIB} peptide was synthesized C-terminal to the gp120 C4 region of the T helper cell determinant (KQIINMWQEVGKAMYA) as described (Lanzavecchia, Nature 393:413-414 (1998)) and the V3_{89.6P} was synthesized C-terminal to the C4_{E9V} (KQIINMWQVVGKAMYA) sequence. Synthetic peptides containing the HIV-1IIIB and SHIV-1 89.6P gp120 V3 loop H-2D^{d}-restricted CTL epitopes were utilized for in vitro restimulation of CTL effector cells and labeling of CTL target cells. For assays with mice immunized with the C4-V3_{IIIB} peptide, peptide R10I (RGPGRAFVTI) was utilized. For assays with mice immunized with the C4-V3_{89.6P} peptide, peptide R16 (RERLSIGPGRAFYARR) was used.

Preparation of murine α₂M*-HIV-1 peptide complexes (α₂M*-HIV peptide): Murine α₂M*-HIV-1 peptide complexes were prepared as previously described (Chu et al, Journal of Immunology 152:1538-1545 (1994)). Briefly, murine α₂M was treated with 100 mM ammonium bicarbonate overnight. The next day the sample was desalted using a PD-10 column (Amersham Pharmacia Biotech) equilibrated in 50 mM Tris-HCl, 100 mM NaCl, pH 7.4. To prepare the complex, this activated α₂M* and HIV-1 peptide were incubated at 50° C. (molar ratio α₂M*:molar ratio peptide 1:40). After 5 h the complex was purified by gel filtration using a Sephacryl S-300HR (Amersham Pharmacia Biotech) column equilibrated in 50 mM Tris-HCl, 100 mM NaCl, pH 7.4. The high molecular weight fractions were pooled, analyzed for HIV-1 peptide incorporation by amino acid analysis, and concentrated by use of CentriPrep® concentrators. Each mole of α₂M* contained ca. 3.2-6.5 moles of HIV-1 peptide. The final stock solutions of α₂M*-HIV-1 peptide contained 3 mg/ml α₂M* and <100 pg/ml endotoxin.

Formulation of peptides with adjuvant: Lyophilized HIV-1 peptides stored at 4° C. were reconstituted in normal saline, and formulated in four groups: 1) C4-V3 peptides in a dose range of 100 μg, 50 μg, 10 μg, and 1 μg per animal were mixed in an emulsion in CFA or IFA (Sigma Chemical Co., St. Louis, Mo.) in a 1:1 volume ratio of peptide in saline to CFA (first immunization), or IFA (subsequent immunizations); 2) C4-V3 peptides in a dose range of 100 μg, 50 μg, 10 μg, and 1 μg per animal were mixed in an emulsion with 20 μg of MPL-SE (Corixa; Hamilton, Mont.) and 10 μg GM-CSF (BioSource International, Camarillo, Calif.); 3) C4-V3 peptides in a dose range of 50 μg, 10 μg, 5 μg, 1 μg, 0.5 μg and 0.1 μg per animal coupled to α₂M* as described previously (Chu et al, Journal of Immunology 152:1538-1545 (1994)); and 4) C4-V3 peptide in a dose range of 10 μg, 5 μg, 1 μg, 0.5 μg and 0.1 μg per animal coupled to α₂M* and formulated in 20 μg of MPL-SE and 10 μg GM-CSF.

Immunizations: Female Balb/C mice, 6-8 weeks of age, were obtained from Charles River Laboratories (Raleigh, N.C.). Three mice were used for each peptide dose group. Each animal was injected subcutaneously (SQ) in 4 sites under the front legs and thighs with specified immunogen formulation in a total volume of 0.4 ml. Mice were injected with immunogens on day 0, 14 and 28. Serum samples were collected before immunization and 7 days after the final immunization, heat-inactivated (56° C., 45 min), and stored at −20° C. until assayed. For CTL assays, spleens were harvested 12-15 days after the final immunization and splenocytes prepared using standard methodology.

ELISA assays: High-binding flat-bottom 96-well microtiter plates (Costar; Corning, N.Y.) were used for all assays. Plates were coated overnight at 4° C. with 50 ng/well C4-V3 peptides in a total volume of 0.05 ml carbonate coating buffer (carbonate buffer, pH 9.6, 0.05% sodium azide). Before use, plates were blocked for non-specific binding with 2% BSA in coating carbonate buffer. Serum samples were serially diluted 2-fold in 0.2% Tween-20 PBS. Pre-immune mouse sera were used as negative controls. Antibody titers against immunizing peptide were performed in standard ELISA assays (Yancopoulos et al, Cell 93:661-664 (1998)). To assay the reactivity with α₂M* itself of antisera from mice immunized with α₂M*-C4-V3_{IIIB} peptide, 96-well plates were coated with α₂M*, hepatitis B surface antigen (HBsAg) coupled to α₂M* (Salvesen et al, Biochem. J. 187:695-699 (1980)) or C4-V3_{IIIB} coupled to α₂M* as described above. The antibody end-point binding titers were determined as the reciprocal of the highest dilution of the serum assayed against corresponding peptides or proteins giving an absorbance₄₅₀ₙₘ of experiment/control (E/C) of ≧3.0.

CTL Assay: Restimulation of effector cells: Splenocytes were separated using lymphocyte separation medium (ICN Biomedicals Inc.; Aurora, Ohio) and used as effector cells to monitor HIV-specific CTL responses. Splenocytes (1×10⁷ cells/ml) were resuspended in CTL media (RPMI 1640, 10% FBS, HEPES, Pen/Strep, 2-mercaptoethanol, 2 ml 2N NaOH, sodium pyruvate) and added to a 24-well plate (750 μl/well) followed by the addition of CTL media containing the appropriate CTL epitope peptide (final peptide concentration, 1 μg/ml) for in vitro stimulation. Splenocytes from naïve mice served as controls. On day 3, 500 μl of CTL media containing recombinant murine IL-2 (rmIL-2) was added to CTL effector cells (final concentration of 10 IU/ml rmIL-2). Chromium release assay: P815 cells (H-2D^{d}) (5×10⁵ cells/ml) were labeled with ⁵¹Cr(100 μl/ml of cells; ⁵¹Cr at 1 mCi/ml). To test for peptide-specific lytic activity, cells were incubated with the appropriate CTL epitope peptide (40 μg/ml). Control P815 cells were not labeled with peptide. P815 cells were incubated for 4 hours (37° C. and 10% CO₂). Washed ×3 with CTL media, 5000 cells were added to each well of a 96 well plate. Effector splenocytes were added to targets in a wide effector:target (E:T) ratio. Spontaneous ⁵¹Cr release and maximum release were determined as described (Jackson et al, FASEB 11:457-465 (1997)). Percent specific lysis was calculated as follows: ([experimental CPM]−[spontaneous CPM])÷(maximum CPM spontaneous CPM)×100. Results are presented as peptide-specific lysis % calculated by subtracting the percent specific lysis of control target cells from the percent specific lysis of the peptide-pulsed target cells at the same E:T ratio.

Antibody Isotyping: ELISA assays of mouse serum samples were performed as above except that the secondary antibody (biotin-labeled rabbit anti-mouse) was replaced with either biotin-labeled rat anti-mouse IgG₁ (BioSource; Camarillo Calif.; 1:1000), biotin-labeled rat anti-mouse IgG₂ₐ (BioSource; 1:1000), or biotin-labeled rat anti-mouse IgG_{2b} (BioSource; 1:1000).

### Results

Antibody responses to HIV envelope C4-V3_{IIIB} peptides using α₂M*-HIV peptide as immunogen. The frequency of serum antibody responses in Balb/c mice immunized with HIV envelope peptide C4-V3_{IIIB}, either coupled to α₂M* (α₂M*-HIV peptide), using CFA/IFA as a positive adjuvant control, or using peptide alone with no adjuvant, are summarized in Table 3. Immunization of Balb/c mice with C4-V3_{IIIB} at doses of 50 μg or 100 μg using no adjuvant resulted in antibody responses with antibody titers of 1:800 and 1:3,200 in 2 of 3 animals, respectively. Antibody titers in mice receiving 50 μg or 100 μg of C4-V3_{IIIB} peptide in CFA/IFA were significantly increased (GMT of 9,600×/÷3,200 to 20,266×/÷15,494, p<0.05) (Table 3). No detectable antibody responses were seen in animals immunized with 10 μg or less of C4-V3_{IIIB} peptide using either CFA/IFA or no adjuvant (Table 3).

TABLE 3
Comparison of the Ability of HIV gp120 C4-V3_{IIIB} Peptide To Induce Antibodies In Balb/C Mice Using
Murine α₂M* and CFA/IFA

| | Number of Animal Responding/Number of Animal Injected with Dose Range of C4-V3_{IIIB} Peptide | | | | | | |
|---|---|---|---|---|---|---|---|
| | (ELISA End-Point Titer†) | | | | | | |
| Adjuvant | 100 μg | 50 μg | 10 μg | 5 μg | 1 μg | 0.5 μg | 0.1 μg |
| Murine | ND | 6/6 | 9/9 | 9/9 | 9/9 | 6/9 | 3/6 |
| α₂M* | | (800-6,400) | (400-51,200) | (200-12,800) | (50-3,200) | (50-1,600) | (100-200) |
| CFA + IFA | 3/3 | 3/3 | 0/3 | ND | ND | ND | ND |
| | (3,200-12,800) | (3,200-51,200) | (<50) | | | | |
| None | 2/3 | 2/3 | 0/3 | ND | 0/3 | ND | ND |
| | (800-1,600) | (800-3,200) | (<50) | | (<50) | | |

†Data represent ELISA endpoint titers of mouse sera as the reciprocal of the highest dilutions of serum samples at which the E/C was ≧3.0 in anti-immunizing peptide ELISA after three immunizations. ND = Not done.
Note: Where α₂M* is indicated as the adjuvant, the α₂M* is covalently coupled to the peptide.

In contrast, immunization of mice with 50 μg, 10 μg, and 1 μg of C4-V3_{IIIB} peptide coupled to α₂M* resulted in sero-conversion (defined as titer ≧1:50) in all 33 animals tested (Table 3). Six of 9 animals immunized with 0.5 μg of C4-V3_{IIIB} peptide and 3 of 6 animals immunized with as low as 0.1 μg of C4-V3_{IIIB} peptide coupled to α₂M* resulted in sero-converted (Table 3). Mice immunized with 10 μg of C4-V3_{IIIB} peptide coupled to α₂M* had high antibody responses (GMT of 12,977×/÷5,867) that were equivalent to antibody responses induced by 100 μg or 50 μg of C4-V3_{IIIB} peptide (GMT of 9,600×/÷3,200 to 20,266)×/÷15,499) in CFA/IFA (p>0.6). Because 50 μg of C4-V3_{IIIB} peptide coupled to α₂M* did not augment antibody levels above levels in mice that received 10 μg of C4-V3_{IIIB} peptide coupled to α₂M*, 10 μg was the highest dose of C4-V3 peptide coupled to α₂M* that was used in subsequent experiments.

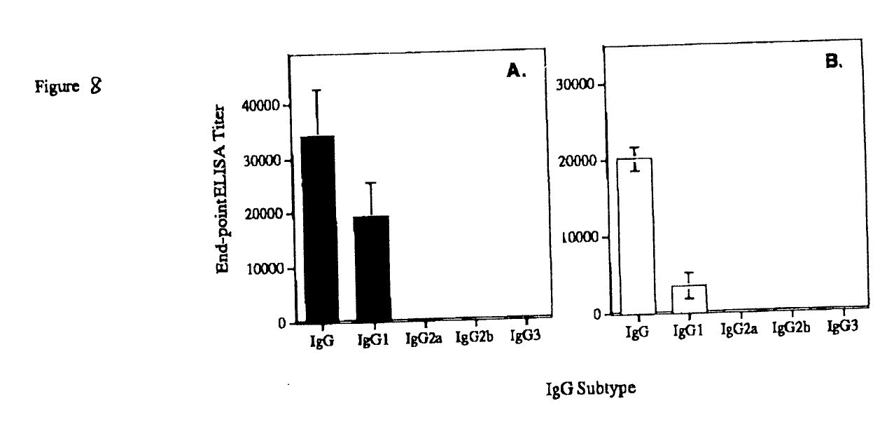

Antisera from mice receiving C4-V3_{IIIB} peptide coupled to α₂M* or in CFA/were assayed by ELISA to determine the immunoglobulin isotypes of their anti-HIV antibodies. The primary isotype of anti-HIV C4-V3_{IIIB} antibody in animals immunized with C4-V3_{IIIB} peptide coupled to α₂M* was IgG₁, similar to that in animals immunized with C4-V3_{IIIB} peptide using CFA/IFA (FIG. 8). These results indicate that a similar Th₂-type of humoral immune response is generated with HIV subunit peptide in α₂M* as is induced with HIV peptide in CFA/IFA.

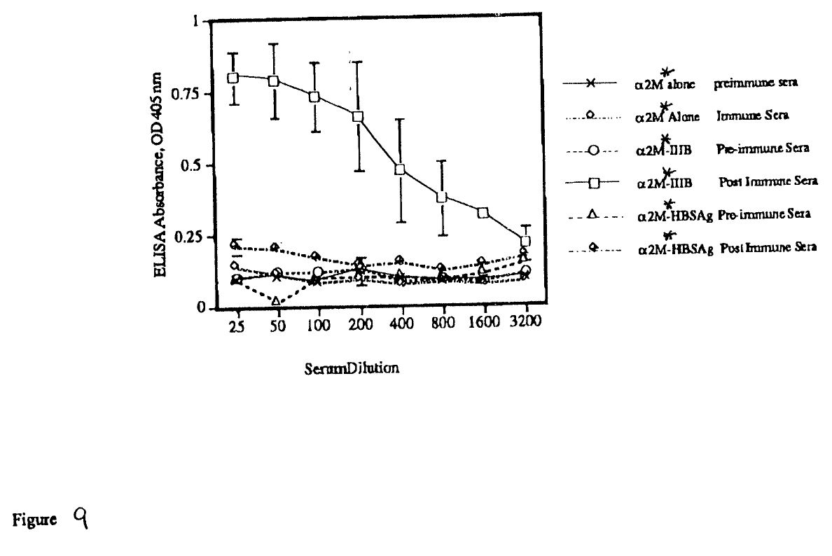

Assay of immunized mouse sera for anti-α₂M* antibodies: To determine if antisera raised in mice by C4-V3_{IIIB} coupled to α₂M* reacted with α₂M* itself, antisera from mice receiving C4-V3 peptide coupled to α₂M* were assayed by ELISA for their reactivity to α₂M*, HBsAg coupled to α₂M*, or with C4-V3_{IIIB} coupled to α₂M*. As shown in FIG. 9, none of pre-immune sera reacted with α₂M*, with HBsAg coupled to α₂M*, or with C4-V3_{IIIB} coupled to α₂M*. Post-immune sera raised by C4-V3_{IIIB} coupled to α₂M* only reacted with C4-V3_{IIIB} coupled to α₂M*, but did not react with α₂M* itself nor with HBsAg coupled to α₂M* (FIG. 9). These results demonstrated that murine α₂M* was not immunogenic in mice when coupled to immunogens.

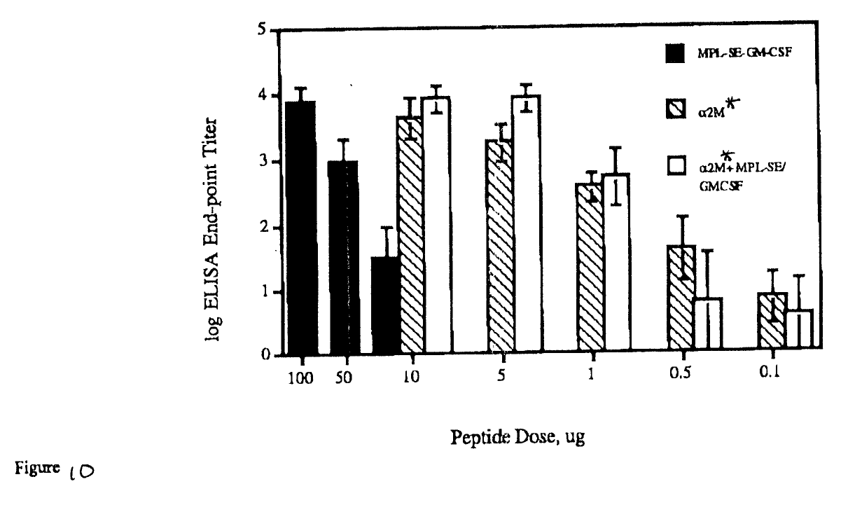

Comparison of MPL-SE/GM-CSF with murine α₂M* as adjuvants: Similar to CFA/IFA, MPL-SE/GM-CSF only enhanced antibody responses with high doses (100 μg or 50 μg) of C4-V3_{IIIB} peptide immunogen (FIG. 10). MPL-SE/GM-CSF as adjuvant induced a maximal antibody response with GMT of 7,352×/÷9,307 with 100 μg of C4-V3_{IIIB} peptide, but did not significantly enhance immune responses with 10 μg or less of C4-V3_{IIIB} peptide (FIG. 10).

In contrast, the adjuvant effect of MPL-SE/GM-CSF was significantly enhanced when MPL-SE/GM-CSF was formulated with 10 μg or less C4-V3_{IIIB} peptide coupled to α₂M* (p<0.005) (FIG. 10). For example, the combination of C4-V3_{IIIB} peptide coupled to α₂M* and MPL-SE/GM-CSF induced maximal antibody responses (GMT of 1:8,123×/÷3,200) with 10 μg or 5 μg of C4-V3_{IIIB} peptide. This MPL-SE/GM-CSF/α₂M* adjuvant formulation of C4-V3_{IIIB} peptide decreased by 20-fold the C4-V3_{IIIB} peptide dose required to induce maximal antibody responses, compared to the amount of peptide needed to achieve equivalent antibody responses using MPL-SE/GM-CSF.

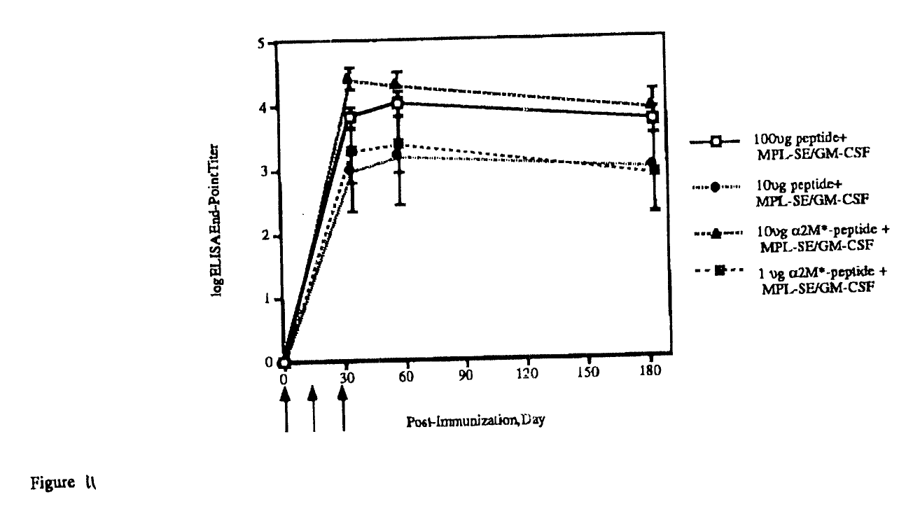

A determination was also made as to whether the antibody response induced with C4-V3_{IIIB} peptide using MPL-SE/GM-CSF or using MPL-SE/GM-CSF/α₂M* adjuvant formulation was long-lasting. The antibody titers of mice immunized with C4-V3_{IIIB} peptide using MPL-SE/GM-CSF/α₂M* adjuvant formulation remained relative stable through 4 months after the final immunization (FIG. 11). At day 184 (129 days after the third injection), the GMT of mice receiving 10 μg and 1 μg of α₂M*-peptide+MPL-SE/GM-CSF were 14,933×/÷9,776 and 2,683×/÷3,311, respectively, and were similar to the GMT of mice receiving 100 μg and 10 μg of C4-V3_{IIIB} peptide with adjuvant MPL-SE/GM-CSF (6,400×/÷5,543 and 4816×/÷6,957, respectively) (>0.5).

To determine the reproducibility of the enhanced immunogenicity of α₂M*-HIV peptide and combination of α₂M*-HIV-1 peptide with MPL-SE/GM-CSF, a different HIV-1 envelope immunogen, the simian human immunodeficiency virus (SHIV) envelope C4-V3_{89.6P} peptide, was tested. The C4-V3_{89.6P} peptide has been proven to be very immunogenic (Aruffo et al, Cell 61:1303-1313, Hieshima et al, J. Biol. Chem. 272:5846 (1997)), and is consistently more immunogenic than C4-V3_{IIIB} peptide.

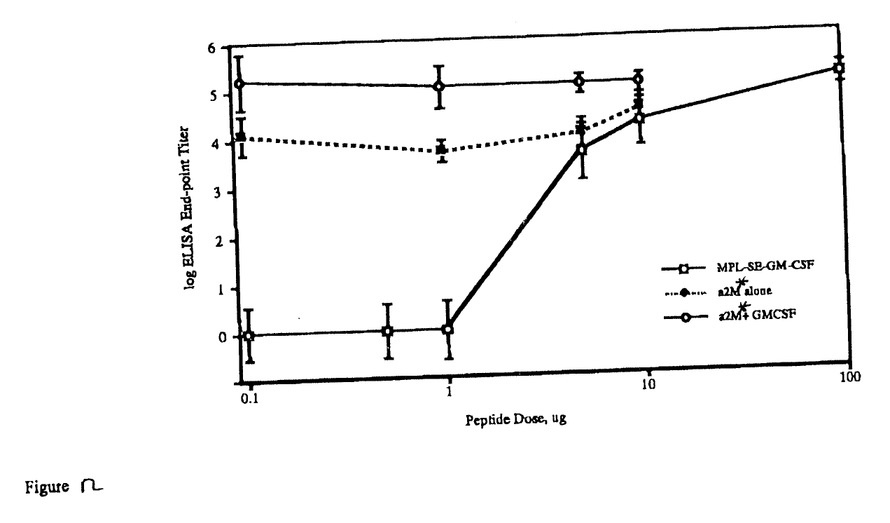

Immunization of Balb/c mice with C4-V3_{89.6P} peptide alone at doses of 100 and 10 μg induced antibody responses with GMT of 12,882×/÷7,466, and 1:158×/÷33, respectively (FIG. 12). MPL-SE/GM-CSF as an adjuvant combination significantly augmented antibody responses induced by C4-V3_{89.6P} peptide at doses of 100 μg (p<0.05) and 10 μg (p<0.001) (FIG. 12). Similar to C4-V3_{IIIB} peptide, 100 μg of C4-V3_{89.6P} peptide induced the highest antibody responses with GMT of 1:128,224×/÷34,133 (FIG. 12). Immunization with 10 μg and 5 μg of C4-V3_{89.6P}, using MPL-SE/GM-CSF as an adjuvant, induced GMT of 16,218×/÷4,266 and 4,043×/÷3,466, respectively. Immunization with 1 μg and 0.5 μg of C4-V3_{89.6P} using MPL-SE/GM-CSF as an adjuvant combination induced no detectable antibody responses (FIG. 12).

In contrast, complexes of α₂M* with C4-V3_{89.6P} peptide enhanced antibody responses at wide dose ranges of peptide (FIG. 12). C4-V3_{89.6P} peptide coupled to α₂M*, at doses as low as 0.1 μg, induced antibody responses with GMT of 1:12,882×/÷5,644. To achieve this same level of antibody response required immunization with 1,000-fold higher amounts of immunogen alone or 100-fold higher amounts of immunogen using MPL-SE/GM-CSF as adjuvant (FIG. 12).

The combination of α₂M*-C4-V3_{89.6P} peptide with MPL-SE/GM-CSF not only decreased the required dose of C4-V3_{89.6P} peptide for maximal antibody response, but also induced higher antibody titers than MPL-SE/GM-CSF or α₂M* alone (GMT of 102,329×/÷45,154 to 162,181×/÷34,133) throughout all dose ranges (FIG. 12). In comparison with α₂M*-C4-V3_{89.6P} peptide alone as immunogen, the combination of MPL-SE/GM-CSF with α₂M*-C4-V3_{89.6P} peptide significantly increased antibody responses induced by C4-V3_{89.6P} at doses of 5 μg (p<0.04), 1 μg (p<0.02) and 0.1 μg (p<0.01) (FIG. 12).

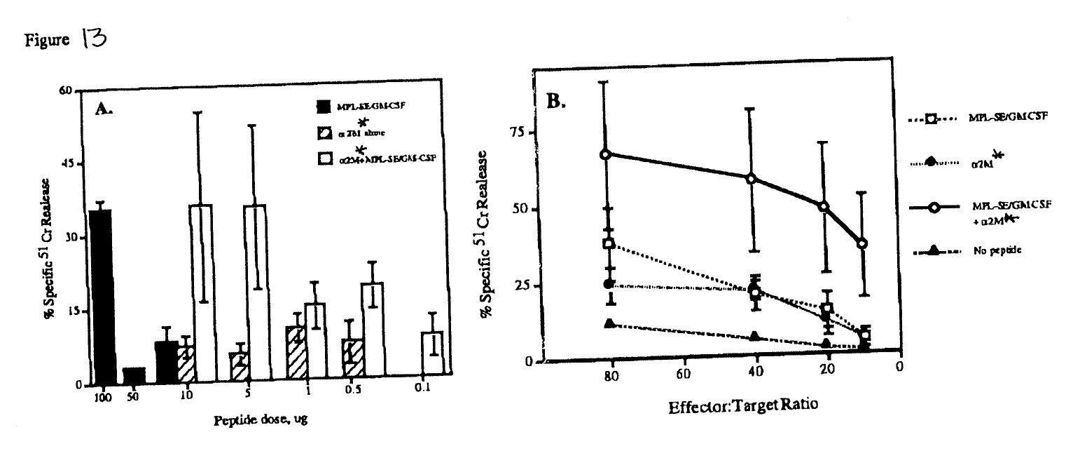

CTL responses to HIV envelope C4-V3 peptides using α₂M* or MPL-SE/GM-CSF as adjuvants: It was next determined whether CTL responses would be induced by immunization of Balb/c mice with C4-V3 peptides using either α₂M*, MPL-SE/GM-CSF or the combination of α₂M* and MPL-SE/GM-CSF as adjuvants. The specific cell lysis induced by 10 μg (37%±19%) or 5 μg (35%±2.9%) of C4-V3_{IIIB} peptide using the combination of α₂M*-C4-V3_{IIIB}+MPL-SE/GM-CSF was only equivalent to that (35%±3.6%) induced by 100 μg of C4-V3_{IIIB} peptide using MPL-SE/GM-CSF alone as adjuvant (FIG. 13A). Shown in FIG. 13B is the comparison of CTL responses induced by immunization of mice with 10 μg of C4-V3_{IIIB} peptide using α₂M*-HIV peptidealone, MPL-SE/GM-CSF alone, or combinations of α₂M*-HIV peptidewith MPL-SE/GM-CSF. Combinations of α₂M*-HIV peptide with MPL-SE/GM-CSF resulted in the highest percentage of specific cell lysis when 10 μg C4-V3_{IIIB} peptide was used for immunization (FIG. 13). Similarly, the combination of α₂M*-HIV peptide with MPL-SE/GM-CSF also resulted in induction of significant CTL responses to C4-V3_{89.6P} peptides.

All documents cited above are hereby incorporated in their entirety by reference.

One skilled in the art will appreciate from a reading of this disclosure that various changes in form and detail can be made without departing from the true scope of the invention.

What is claimed is:

1. A composition comprising activated alpha-2-macroglobulin (α₂M*), 3-O-deacylated monophosphoryl lipid A (MPL) and granulocyte macrophage colony stimulating factor (GM-CSF).

2. The composition according to claim 1 wherein said composition further comprises a biomolecule covalently bound to said α₂M*.

3. The composition according to claim 2 wherein said biomolecule is selected from the group consisting of a peptide, polypeptide or protein, a carbohydrate or a nucleic acid.

4. The composition according to claim 3 wherein said biomolecule is a peptide, polypeptide or protein.

5. The composition according to claim 4 wherein said peptide, polypeptide or protein is a bacterial or viral peptide, polypeptide or protein.

6. The composition according to claim 5 wherein said peptide, polypeptide or protein is a viral peptide, polypeptide or protein.

7. The composition according to claim 6 wherein the peptide, polypeptide or protein is a human immunodeficiency virus (HIV), influensa, tuberculosis, Ebola virus, hepatitis C, hepatitis B, measles, mumps, polio, tetanus or malarial peptide, polypeptide or protein.

8. The composition according to claim 7 wherein said peptide, polypeptide or protein is a HIV peptide, polypeptide or protein.

9. The composition according to claim 1 wherein said composition further comprises a polyvalent HIV immunogen covalently bound to said α₂M*.

10. The composition according to claim 9 wherein said polyvalent immunogen comprises about 50-100 HIV peptides.

11. The composition according to claim 1 or 2 wherein said composition further comprises at least one molecule selected from the group consisting of a cytokine, a chemokine, a B cell activator or growth factor and an angiogenic factor.

12. The composition according to claim 11 wherein said composition comprises a chemokine selected from the group consisting of Thymus and Activation Regulated Chemokine (TARC), Epstein-Barr Virus-induced molecule 1 (EBI-1) Ligand Chemokine (ELC), Liver and Activation Regulated Chemokine (LARC), B Lymphocyte Chemokine (BLC) and MDC (Macrophage Derived Chemokine).

13. The composition according to claim 11 wherein said composition comprises a cytokine selected from the group consisting of IL-2, IL-15, IL-7 and IL-12.

14. The composition according to claim 11 wherein said composition comprises an angiogenic factor selected from the group consisting of vascular endothelial growth factor (VEGF), basic fibroblast growth factor (bFGF) and low molecular weight hyaluronan fragment.

15. The composition according to claim 11 wherein said composition comprises a B cell activator or growth factor selected from the group consisting of B lymphocyte stimulator (BLyS) and proliferation-inducing ligand (APRIL).

16. The composition according to claim 1 wherein said MPL is present as a stable emulsion.

17. As immunogenic composition comprising at least one immunogen and the composition according to claim 1, wherein said immunogen is covalently bound to said α₂M*.

18. The immunogenic composition according to claim 17 further comprising at least one molecule selected from the group consisting of a cytokine, a chemokine, a B cell activator or growth factor and an angiogenic factor.

19. The immunogenic composition according to claim 17 wherein said immunogenic composition is in a form suitable for oral, vaginal or intranasal administration.

20. The immunogenic composition according to claim 17 wherein said immunogenic composition is in a form suitable for administration by injection.

21. A method of eliciting an immune response in a mammal comprising administering to said mammal an amount of the composition according to claim 2 sufficient to elicit said response.

22. A composition comprising MPL and GM-CSF and a chemokine.

23. The composition according to claim 22 wherein said composition further comprises a biomolecule other than GM-CSF.

24. The composition according to claim 23 wherein said biomolecule is selected from the group consisting of a peptide, polypeptide or protein, a carbohydrate or a nucleic acid.

25. The composition according to claim 24 wherein said biomolecule is a peptide, polypeptide or protein.

26. The composition according to claim 25 wherein said peptide, polypeptide or protein is a bacterial or viral peptide, polypeptide or protein.

27. The composition according to claim 26 wherein said peptide, polypeptide or protein is a viral peptide, polypeptide or protein.

28. The composition according to claim 27 wherein the peptide, polypeptide or protein is a human immunodeficiency virus (HIV), influensa, tuberculosis, Ebola virus, hepatitis C, hepatitis B, measles, mumps, polio, tetanus or malarial peptide, polypeptide or protein.

29. The composition according to claim 28 wherein said peptide, polypeptide or protein is a HIV peptide, polypeptide or protein.

30. The composition according to claim 29 wherein said composition comprises about 50-100 HIV peptides.

31. The composition according to claim 22 or 23 wherein said composition further comprises at least one cytokine, B cell activator or growth factor or angiogenic factor.

32. The composition according to claim 22 wherein said chemokine is selected from the group consisting of TARC, ELC, LARC, BLC and MDC.

33. The composition according to claim 22 wherein said composition further comprises a cytokine selected from the group consisting of IL-2, IL-15, IL-7 and IL-12.

34. The composition according to claim 22 wherein said composition further comprises an angiogenic factor selected from the group consisting of VEGF, bFGF and low molecular weight hyaluronan fragment.

35. The composition according to claim 22 wherein said composition further comprises a B cell activator or growth factor selected from the group consisting of BLyS and APRIL.

36. The composition according to claim 22 wherein said MPL is present as a stable emulsion.

37. An immunogenic compsition comprising at least one immunogen and the composition according to claim 22.

38. The immunogenic composition according to claim 37 further comprising at least one molecule selected from the group consisting of a cytokine other than GM-CSF, a B cell activator or growth factor and an angiogenic factor.

39. The immunogenic composition according to claim 37 wherein said immunogenic composition is in a form suitable for oral, vaginal or intranasal administration.

40. The immunogenic composition according to claim 37 wherein said immunogenic composition is in a form suitable for administration by injection.

41. A method of eliciting an immune response in a mammal comprising administering to said mammal an amount of the composition according to claim 23 sufficient to elicit said response.
